(19) United States
(12) Patent Application Publication
(10) Pub. No.: US 2023/0021051 A1
(43) Pub. Date: Jan. 19, 2023

(54) Composition Containing Black Ginger Extract and Composition for Oral Administration

(71) Applicant: Maruzen Pharmaceuticals Co., Ltd., Hiroshima (JP)

(72) Inventors: Takashi Tagawa, Hiroshima (JP); Jun Ogasawara, Hiroshima (JP)

(21) Appl. No.: 17/780,590

(22) PCT Filed: Oct. 16, 2020
(86) PCT No.: PCT/JP2020/039146

§ 371 Date: May 27, 2022

(30) Foreign Application Priority Data
Dec. 26, 2019 (JP) 2019-236771

(51) Int. Cl. A61K 36/906; A61K 47/61

(52) U.S. Cl. CPC A61K 36/906; A61K 47/61; A61K 2236/331

(57) ABSTRACT

A composition containing black ginger extract including black ginger extract, and cyclodextrin. The composition containing black ginger extract includes certain 6 polymethoxyflavones (6PMF). When a total amount of 6PMF in the composition containing black ginger extract is determined as 100 parts by mass, an amount of 5-hydroxy-7-methoxyflavone in the composition containing black ginger extract is 5 parts by mass or less. When absorbance of an aqueous solution of the composition containing black ginger extract, in which a total amount of the 6 polymethoxyflavones is adjusted to a certain amount, is measured, the absorbance of the aqueous solution satisfies P1/V1≥1.45.

### TECHNICAL FIELD

The present invention relates to a composition containing black ginger extract, and a composition for oral administration that contains the composition containing black ginger extract.

### BACKGROUND ART

Black ginger (Thai black ginger) is a plant native to Thailand. The black ginger has been traditionally used as a folk medicine for nutritional enhancement, such as increasing energy, enhancing sexual performance, improving symptoms of stomach or bowel illness, and releasing pain. In some regions of South Asia, the black ginger is sliced and brewed to make a tea, and people drink the tea for improving heath. Also, the black ginger is immersed in liquors, and people drink it as a medical liquor. Therefore, the black ginger is highly safe to intake.

Moreover, it has been known that black ginger extract exhibits excellent an anti-oxidant effect, anti-aging effect, anti-inflammatory effect, hair growth effect, anti-obesity effect, skin-brightening effect, and the like (see PTL 1). Therefore, there has been an attempt that an active ingredient is extracted from black ginger, and the black ginger extract having high activities is added to a composition for oral administration to administer the composition.

However, the black ginger extract has the following problem. The black ginger extract does not give a sweet taste, and has an unpleasant taste, such as bitterness and astringency. Therefore, the black ginger extract leaves an unpleasant aftertaste and it is difficult to drink repeatedly.

To solve the above-described problem, proposed is a black ginger extract composition, with which the taste the black ginger extract has, such as bitterness, astringency, and aftertaste, has been improved (see PTL 2).

However, the improvement of the taste made with the proposed black ginger extract composition is not sufficiently satisfactory. Moreover, the proposed black ginger extract composition is not sufficiently soluble to water and an acid solution. Therefore, the proposed black ginger extract composition cannot be easily applied for a composition for oral administration.

Accordingly, it is currently strongly desired to develop a composition containing black ginger extract, with which the taste of the black ginger extract is further improved, and solubility of the black ginger extract to water and an acid solution is improved, and a composition for oral administration including the composition containing black ginger extract.

### CITATION LIST

### Patent Literature

PTL 1: Japanese Patent Application Laid-Open (JP-A) No. 2009-051790
PTL 2: JP-A No. 2013-192513

### SUMMARY OF INVENTION

### Technical Problem

The present invention aims to solve the above existing problems in the art and achieve the following objects.

Specifically, an object of the present invention is to provide a composition containing black ginger extract, with which a taste black ginger extract has is improved and solubility to water and an acid solution is improved, and a composition for oral administration that contains the composition containing black ginger extract.

### Solution to Problem

### <1> A Composition Containing Black Ginger Extract, Including:

black ginger extract; and
cyclodextrin,
wherein the composition containing black ginger extract includes
6 polymethoxyflavones including 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, and 3,5,7,4′-tetramethoxyflavone,
when a total amount of the 6 polymethoxyflavones in the composition containing black ginger extract is determined as 100 parts by mass, an amount of 5-hydroxy-7-methoxyflavone in the composition containing black ginger extract is 5 parts by mass or less,
when absorbance of an aqueous solution of the composition containing black ginger extract, in which a total amount of the 6 polymethoxyflavones is adjusted to 0.006 mg/mL, is measured, the absorbance of the aqueous solution satisfies Formula (1) below

P1/V1≥1.45 Formula(1)

where, in Formula (1), V1 is the minimum value of absorbance in a wavelength range of from 230 nm to 240 nm, and P1 is the maximum value of absorbance in a wavelength range of from 260 nm to 270 nm.
<2> a Composition for Oral Administration Including the composition containing black ginger extract according to <1>.

### Advantageous Effects of Invention

The present invention can solve the above-described various problems existing in the art, and can achieve the above-described object. The present invention can provide a composition containing black ginger extract, with which a taste black ginger extract has is improved and solubility to water and an acid solution is improved, and a composition for oral administration that contains the composition containing black ginger extract.

### BRIEF DESCRIPTION OF DRAWINGS

FIG. 1 is a graph depicting a UV absorption spectrum obtained in Test Example 2 by measuring absorbance of the composition containing black ginger extract aqueous solution of Example 1, in which the total amount of 6PMF is adjusted to 0.006 mg/mL. “V1” is the minimum value of the absorbance in the wavelength range of from 230 nm to 240 nm, “P1” is the maximum value of the absorbance in the wavelength range of from 260 nm to 270 nm, “V2” is the minimum value of the absorbance in the wavelength range of from 275 nm to 290 nm, and “P2” is the maximum value of the absorbance in the wavelength range of from 320 nm to 340 nm.

### DESCRIPTION OF EMBODIMENTS

### (Composition Containing Black Ginger Extract)

The composition containing black ginger extract of the present invention includes at least black ginger extract and cyclodextrin, and may further include other ingredients according to the necessity.

### <Black Ginger Extract>

Black ginger (Thai black ginger) used as an extraction raw material of the black ginger extract is a plant belongs to the genus Kaempferia of the family Zingiberaceae. The scientific name of the black ginger is Kaempferia parviflora. The black ginger is distributed across Thai etc., in Southeaste Asia, and can be readily sourced from this region.

A method for sourcing the black ginger is not particularly limited, and may be appropriately selected depending on the intended purpose. The black ginger may be collected by harvesting black ginger grown in the nature. Alternatively, a commercial product may be used.

### <<6 Polymethoxyflavone (6PMF)>>

The composition containing black ginger extract includes 6 polymethoxyflavones including 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, and 3,5,7,4′-tetramethoxyflavone (the 6 polymethoxyflavones may be referred to as “6PMF” hereinafter).

The 6 polymethoxyflavones are substances derived from the black ginger extract.

5,7,3′,4′-tetramethoxyflavone is the compound represented by Structural Formula (1) below.

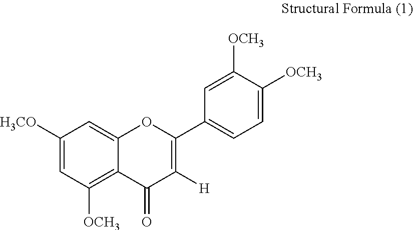 [SMILES: COc1cc(OC)c2c(=O)cc(-c3ccc(OC)c(OC)c3)oc2c1]

3,5,7,3′,4′-pentamethoxyflavone is the compound represented by Structural Formula (2) below.

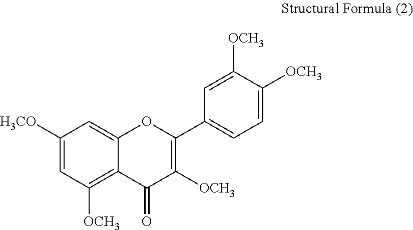 [SMILES: COc1cc(OC)c2c(=O)c(OC)c(-c3ccc(OC)c(OC)c3)oc2c1]

5,7-dimethoxyflavone is the compound represented by Structural Formula (3) below.

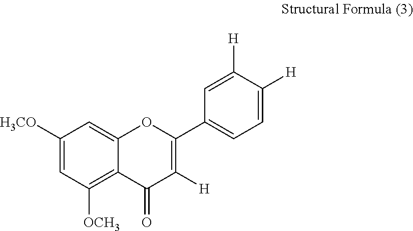 [SMILES: COc1cc(OC)c2c(=O)cc(-c3ccccc3)oc2c1]

5,7,4′-trimethoxyflavone is the compound represented by Structural Formula (4) below.

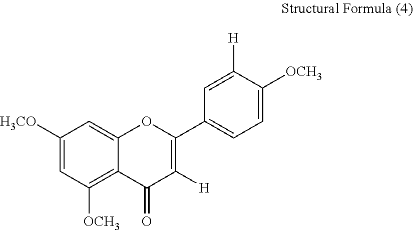 [SMILES: COc1ccc(-c2cc(=O)c3c(OC)cc(OC)cc3o2)cc1]

3,5,7-trimethoxyflavone is the compound represented by Structural Formula (5) below.

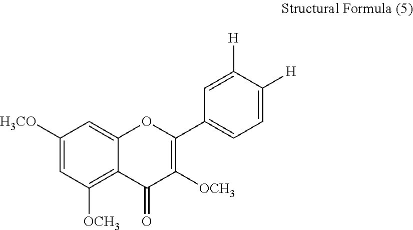 [SMILES: COc1cc(OC)c2c(=O)c(OC)c(-c3ccccc3)oc2c1]

3,5,7′-tetramethoxyflavone is the compound represented by Structural Formula (6) below.

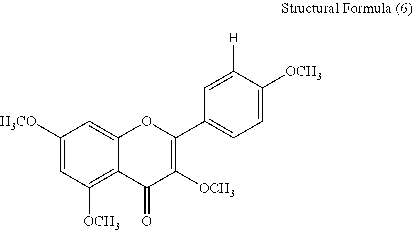 [SMILES: COc1ccc(-c2oc3cc(OC)cc(OC)c3c(=O)c2OC)cc1]

An amount of 6PMF in the composition containing black ginger extract is not particularly limited, and may be appropriately selected depending on the intended purpose. The lower limit of the amount of 6PMF is preferably 1% by mass or greater, and more preferably 5% by mass or greater, relative to the total solid content of the composition containing black ginger extract. Moreover, the upper limit of the amount of 6PMF is preferably 20% by mass or less, and more preferably 15% by mass or less, relative to the total solid content of the composition containing black ginger extract. The upper limit and the lower limit of the amount of 6PMF in the composition containing black ginger extract may be appropriately combined depending on the intended purpose. The amount of 6PMF defined by the upper limit and lower limit thereof is preferably 1% by mass to 20% by mass, and more preferably 5% by mass to 15% by mass, relative to the total solid content of the composition containing black ginger extract.

In the present invention, the total solid content of the composition containing black ginger extract is a mass of solids obtained by freeze-drying the composition containing black ginger extract.

The amount of 6PMF in the composition containing black ginger extract can be measured and quantified by high-performance liquid chromatography (HPLC) under the analysis conditions described in Examples (Preparation Example 2) below.

### <<5-Hydroxy-7-Methoxyflavone (5-HMF)>>

The composition containing black ginger extract may include 5-hydroxy-7-methoxyflavone (may be referred to as “5-HMF” hereinafter).

5-hydroxy-7-methoxyflavone is a substance derived from the black ginger extract, and is the compound represented by Structural Formula (7) below.

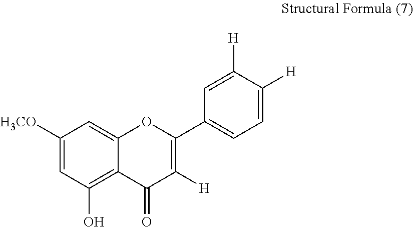 [SMILES: COc1cc(O)c2c(=O)cc(-c3ccccc3)oc2c1]

An amount of 5-HMF in the composition containing black ginger extract is 5 parts by mass or less, preferably 3.5 parts by mass or less, more preferably 3.0 parts by mass or less, and particularly preferably 2.0 parts by mass or less, when a total amount of 6PMF in the composition containing black ginger extract is determined as 100 parts by mass. The amount of 5-HMF being 5 parts by mass or less relative to 100 parts by mass of the total amount of 6PMF is advantageous because the taste the black ginger extract has can be improved, and solubility to water and an acid solution can be improved. When the amount of 5-HMF is greater than 5 parts by mass relative to 100 parts by mass of the total amount of the 6PMF, in contrast, the taste the black ginger extract has cannot be improved, and solubility to water and an acid solution cannot be improved.

The amount of 5-HMF in the composition containing black ginger extract can be measured and quantified by high-performance liquid chromatography (HPLC) under the analysis conditions described in Examples (Preparation Example 2) below.

The composition containing black ginger extract includes various substances derived from the black ginger extract, as well as 6PMF and 5-HMF. Therefore, polymethoxyflavones derived from the black ginger extract, which are other than 6PMF, or hydroxymethoxyflavones derived from the black ginger extract, which are other than 5-HMF, may be included in the composition containing black ginger extract.

### —Extraction Method of Black Ginger Extract—

An extraction method of the black ginger extract is not particularly limited, as long as extract including 6PMF can be obtained. The black ginger extract can be easily obtained by a method typically used for extracting plants. Examples of the extraction method include a method where black ginger used as the extraction raw material is immersed in a processing tank filled with an extraction solvent, which is then optionally appropriately stirred to dissolve soluble substances, followed by subjecting to filtration to remove extraction residues, and purifying the filtrate to obtain black ginger extract.

The part of the black ginger to be used as the extraction raw material is not particularly limited, and may be appropriately selected depending on the intended purpose, as long as the part thereof includes 6PMF. Examples thereof include: aboveground parts, such as flowers, buds, seeds, seed coats, stems, leaves, branches, and branches and leaves; and underground parts such as roots and rhizomes. The above-listed examples may be used alone or in combination. Among the above-listed examples, the part of the black ginger to be used is preferably underground parts, such as roots and rhizomes.

The size of the black ginger to be used as the extraction raw material is not particularly limited, and may be appropriately selected depending on the intended purpose. Examples thereof include an original size as collected, the desired size by which the black ginger is cut, and a size by which the black ginger is finely milled (powdered).

The state of the black ginger to be used as the extraction raw material is not particularly limited, and may be appropriately selected depending on the intended purpose. Examples thereof include an original state as collected, a dried state, a milled state, and a state of a juice. Among the above-listed examples, the dried state is preferable.

A method for tuning the black ginger into the dried state is not particularly limited, and may be appropriately selected depending on the intended purpose. Examples of the method include drying in the sun, and drying with a typical drier.

The method for turning the black ginger into the milled state is not particularly limited, and may be appropriately selected depending on the intended purpose. Examples thereof include milling with, for example, a mixer, a sugar mill, a power mill, a jet mill, or an impact mill.

The method for turning black ginger into the state of a juice is not particularly limited, and may be appropriately selected depending on the intended purpose. Examples thereof include squeezing.

The extraction solvent of the black ginger extract is not particularly limited, and may be appropriately selected depending on the intended purpose, as long as an extract including 6PMF can be obtained. Examples thereof include water, a hydrophilic solvent, or a mixed solvent of water and a hydrophilic solvent.

The water is not particularly limited, and may be appropriately selected depending on the intended purpose. Examples thereof include pure water, tap water, well water, mineral spring water, mineral water, hot spring water, spring water, fresh water, purified water, hot water, ion-exchanged water, a saline solution, a phosphate buffer solution, and a phosphate-buffered saline. The above-listed examples may be used alone, or in combination.

The hydrophilic solvent is not particularly limited, and may be appropriately selected depending on the intended purpose. Examples thereof include: C1-C5 lower alcohol, such as methanol, ethanol, propyl alcohol, and isopropyl alcohol; lower aliphatic ketone, such as acetone, and methyl ethyl ketone; and C2-C5 polyhydric alcohol, such as 1,3-butyleneglycol, propylene glycol and glycerin. The above-listed examples may be used alone, or in combination.

The mixed solvent of the water and the hydrophilic solvent is not particularly limited, and may be appropriately selected depending on the intended purpose. When the lower alcohol is used as the hydrophilic solvent, 1 part by mass to 90 parts by mass of the hydrophilic solvent is preferably used relative to 10 parts by mass of the water. When the lower aliphatic ketone is used as the hydrophilic solvent, 1 part by mass to 40 parts by mass of the hydrophilic solvent is preferably used relative to 10 parts by mass of the water. When the polyhydric alcohol is used as the hydrophilic solvent, 1 part by mass to 90 parts by mass of the hydrophilic solvent is preferably used relative to 10 parts by mass of the water. The above-listed examples may be used alone, or in combination.

Among the above-listed examples, the extraction solvent of the black ginger extract is preferably a mixed solvent of water and ethanol (water-containing ethanol) considering safety and handling.

When the water-containing ethanol is used as the extraction solvent, a concentration of ethanol is not particularly limited, and may be appropriately selected depending on the intended purpose. The concentration of ethanol in the water-containing ethanol is preferably 50% by volume or greater, and more preferably 60% by volume or greater.

An amount of the extraction solvent for use is not particularly limited, and may be appropriately selected depending on the intended purpose. The amount of the extraction solvent is preferably 5 times to 15 times (mass ratio) the amount of the black ginger used as the extraction raw material.

Extraction conditions (extraction time, extraction temperature, atmosphere conditions, such as pressure) of the black ginger extract are not particularly limited, and may be appropriately selected from methods known in the art according to the intended purpose, as long as an extract including 6PMF can be obtained.

When water is used as the extraction solvent, the extraction is typically performed for about 1 hour to about 4 hours at 50° C. to 95° C. When a mixed solvent of water and ethanol is used as the extraction solvent, the extraction is typically performed for about 30 minutes to about 4 hours at 40° C. to 80° C.

A purification method of the black ginger extract is not particularly limited, and may be appropriately selected depending on the intended purpose, as long as a purified product including 6PMF can be obtained. Examples thereof include chromatography (e.g., partition chromatography, adsorption chromatography, ion-exchange chromatography, and size exclusion chromatography), liquid-liquid partition extraction, and membrane separation. The above-listed purification methods may be used alone or in combination. Among the above-listed examples, the black ginger extract is preferably purified by the adsorption chromatography.

An absorbent resin used for the adsorption chromatography is not particularly limited, and may be appropriately selected depending on the intended purpose, as long as a purified product including 6PMF can be obtained. The absorbent resin is preferably an aromatic absorbent resin, or an aromatic modified absorbent resin.

Specific examples of the absorbent resin include: DIAION HP20, DIAION HP21, SEPABEADS SP825L, SEPABEADS SP850, and SEPABEADS SP207 (all available from Mitsubishi Chemical Corporation); and Amberlite XAD-2, Amberlite XAD4, and Amberlite XAD7 (all available from ORGANO CORPORATION).

An amount of the absorbent resin used for purifying the black ginger extract may be appropriately selected depending on the performance of the absorbent resin. The amount of the absorbent resin is preferably 1 part by volume to 50 parts by volume relative to 1 part by mass of the solid content of the black ginger extract.

In the present invention, the solid content of the black ginger extract is a mass of solids obtained by freeze-drying the black ginger extract.

A solvent used for eluting substances derived the black ginger extract, which is has been adsorbed on the adsorbent resin (may be referred to as an “elution solvent” hereinafter) is not particularly limited, and may be appropriately selected depending on the intended purpose, as long as an elution fraction including 6PMF can be obtained. Examples thereof include water, a hydrophilic solvent, and a mixed solvent of water and a hydrophilic solvent. The solvents listed as the extraction solvents of the black ginger extract can be used as the elution solvent.

Among the above-mentioned examples, the elution solvent is preferably a mixed solvent of water and ethanol (water-containing ethanol).

When the water-containing ethanol is used as the elution solvent, the concentration of the ethanol is not particularly limited, and may be appropriately selected depending on the intended purpose, but the concentration thereof is preferably 60% by volume to 100% by volume, and more preferably 75% by volume, because an elution fraction having a high 6PMF content can be obtained.

Among the elution fractions obtained by the elution solvent, only one elution fraction may be used as black ginger extract, or a plurality of the elution fractions may be mixed together and used as black ginger extract, as long as the elution fraction includes 6PMF.

An amount of the solvent used for the purification is not particularly limited, and may be appropriately selected depending on a desired amount of 6PMF. The amount thereof is preferably 1 time to 20 times (volume ratio) the amount of the adsorbent resin.

A state of the black ginger extract is not particularly limited, and may be appropriately selected depending on the intended purpose. For example, the black ginger extract may be the black ginger extract as extracted, a concentrate of the black ginger extract, a dilution of the black ginger extract, or a dried product of the black ginger extract. Moreover, the black ginger extract may be black ginger extract obtained by mixing or dissolving the dried product of the black ginger extract back in water, a hydrophilic solvent, or a mixed solvent of water and a hydrophilic solvent.

The solid content of the black ginger extract in the composition containing black ginger extract is not particularly limited, and may be appropriately selected depending on the intended purpose. The solid content thereof is preferably 1% by mass to 20% by mass, and more preferably 5% by mass to 15% by mass, relative to the total solid content of the composition containing black ginger extract. When the solid content of the black ginger extract is less than 1% by mass, a desired effect of the black ginger extract that is an active ingredient may not be exhibited. When the solid content of the black ginger extract is greater than 20% by mass, it may be difficult to produce the composition containing black ginger extract.

### <Cyclodextrin>

The cyclodextrin is not particularly limited, and may be appropriately selected depending on the intended purpose. Examples of the cyclodextrin includeα-cyclodextrin, β-cyclodextrin, and γ-cyclodextrin. The above-listed examples may be used alone, or in combination. Among the above-listed examples, γ-cyclodextrin is preferably because the composition containing black ginger extract can be uniformly produced.

As the cyclodextrin, cyclodextrin produced by any of known methods may be used, or a commercial product of cyclodextrin may be used.

An amount of the cyclodextrin in the composition containing black ginger extract is not particularly limited, and may be appropriately selected depending on the intended purpose. When a total amount of 6PMF in the composition containing black ginger extract is 1 part by mass, the amount of the cyclodextrin is preferably 4 parts by mass or greater, more preferably 6 parts by mass or greater, and even more preferably 8 parts by mass or greater. The amount of the cyclodextrin being 4 parts by mass or greater relative to 1 part by mass of the total amount of 6PMF is advantageous because the taste the black ginger extract has can be improved, and solubility to water and an acid solution can be improved. The upper limit of the amount of the cyclodextrin is not particularly limited.

### <<Absorbance Ratio (P1/V1)>>

When absorbance of an aqueous solution of the composition containing black ginger extract, in which a total amount of 6PMF is adjusted to 0.006 mg/mL, is measured, the absorbance of the aqueous solution satisfies Formula (1) below.

P1/V1≥1.45 Formula(1)

In Formula (1), V1 is the minimum value of absorbance in a wavelength range of from 230 nm to 240 nm, and P1 is the maximum value of absorbance in a wavelength range of from 260 nm to 270 nm.

In the present specification, therefore, the value calculated by P1/V1 of Formula (1) may be referred to as an “absorbance ratio (P1/V1).”

As described above, the absorbance ratio (P1/V1) is 1.45 or greater, and preferably 1.5 or greater. The absorbance ratio (P1/V1) being 1.45 or greater is advantageous because the taste the black ginger extract has can be improved, and solubility to water and an acid solution can be improved. When the absorbance ratio (P1/V1) is less than 1.45, in contrast, the taste the black ginger extract has cannot be improved, and solubility to water and an acid solution cannot be improved.

Since the taste the black ginger extract has can be improved, and solubility to water and an acid solution can be improved as long as the absorbance ratio (P1/V1) is 1.45 or greater, the upper limit of the absorbance ratio (P1/V1) is not particularly limited, and may be appropriately selected depending on the intended purpose. However, the upper limit thereof is preferably 2.0 or less.

The upper limit and the lower limit of the absorbance ratio (P1/V1) may be appropriately combined depending on the intended purpose. The range of the absorbance ratio (P1/V1) defined by the upper limit and the lower limit is preferably 1.45 or greater but 2.0 or less, and more preferably 1.5 or greater but 2.0 or less.

### <<Absorbance Ratio (P2/V2)>>

When absorbance of an aqueous solution of the composition containing black ginger extract, in which a total amount of 6PMF is adjusted to 0.006 mg/mL, is measured, the absorbance of the aqueous solution preferably further satisfies Formula (2) below.

P2/V2≥1.42 Formula(2)

In Formula (2), V2 is the minimum value of absorbance in a wavelength range of from 275 nm to 290 nm, and P2 is the maximum value of absorbance in wavelength range of from 320 nm to 340 nm.

In the present specification, therefore, the value calculated by P2/V2 of Formula (2) may be referred to as an “absorbance ratio (P2/V2).”

As described above, the absorbance ratio (P2/V2) is preferably 1.42 or greater, more preferably 1.44 or greater, and even more preferably 1.46 or greater. The absorbance ratio (P2/V2) being 1.42 or greater is advantageous because the taste the black ginger extract has can be improved, and solubility to water and an acid solution can be improved.

Since the absorbance ratio (P2/V2) is preferably 1.42 or greater for the purpose of preferably improving the taste the black ginger extract has, or solubility to water and an acid solution, the upper limit of the absorbance ratio (P2/V2) is not particularly limited, and may be appropriately selected depending on the intended purpose. However, the upper limit of the absorbance ratio (P2/V2) is preferably 1.7 or less.

The upper limit and the lower limit of the absorbance ratio (P2/V2) may be appropriately combined depending on the intended purpose. The range of the absorbance ratio (P2/V2) defined by the upper limit and the lower limit is preferably 1.42 or greater but 1.7 or less, and more preferably 1.44 or greater but 1.7 or less.

The total amount of 6PMF of the composition containing black ginger extract in the aqueous solution for measuring the absorbance can be adjusted by adding distilled water to the composition containing black ginger extract to allow the concentration of 6PMF to be 0.006 mg/mL.

The absorbance of the composition containing black ginger aqueous solution, in which the total amount of 6PMF is adjusted to 0.006 mg/mL, can be measured, for example, by means of an ultraviolet-visible spectrophotometer (model number: UV-1800, available from Shimadzu Corporation) with a quartz cell having a light path length of 10 mm charged with the composition containing black ginger extract aqueous solution. V1, P1, V2, and P2 can be determined from UV absorption spectrum obtained from the measurement performed by the ultraviolet-visible spectrophotometer. V1, P1, V2, and P2 are the values that are background-corrected using distilled water as a control sample. Specifically, V1, P1, V2, and P2 are all values obtained by subtracting a value measured by means of an ultraviolet-visible spectrophotometer (model number: UV-1800, available from Shimadzu Corporation) with a quartz cell having a light path length of 10 mm charged with distilled water as a control sample, from the measured value of the composition containing black ginger extract aqueous solution.

### <<Transmittance>>

The transmittance of the composition containing black ginger extract aqueous solution, in which the total amount of 6PMF is adjusted to 0.05 mg/mL, to light having a wavelength of 660 nm is not particularly limited, and may be appropriately selected depending on the intended purpose. The transmittance is preferably 95% or greater, more preferably 97% or greater, even more preferably 99% or greater, and particularly preferably 99.5% or greater.

The transmittance is an index for solubility of the composition containing black ginger extract to water and an acid solution. The higher the value of the transmittance is, the higher solubility of the composition containing black ginger extract to water and an acid solution is.

The total amount of 6PMF of the composition containing black ginger extract in the aqueous solution for measuring the transmittance can be adjusted by adding distilled water or a 0.1% by mass citric acid solution (pH 2.8) to the composition containing black ginger extract to allow the concentration of 6PMF to be 0.05 mg/mL.

For example, the transmittance of the composition containing black ginger extract aqueous solution, in which the total amount of 6PMF is adjusted to 0.05 mg/mL, to light having a wavelength of 660 nm can be calculated from a result, which is obtained by processing the composition containing black ginger extract aqueous solution with ultrasonic waves for 5 minutes, heating the aqueous solution for 20 minutes at 80° C., and cooling the aqueous solution to room temperature (according to JIS Z 8703:1983 standard), followed by measuring by means of an ultraviolet-visible spectrophotometer (model number: UV-1800, available from Shimadzu Corporation) with a quartz cell having a light path length of 10 mm, charged with the cooled aqueous solution. Specifically the transmittance of the aqueous solution to light having a wavelength of 660 nm can be calculated using distilled water or a 0.1% by mass citric acid solution as a control sample, and determining the light transmittance of the water or 0.1% by mass citric acid solution to light having a wavelength of 660 nm as 100%.

A state of the composition containing black ginger extract is not particularly limited, and may be appropriately selected depending on the intended purpose. Examples thereof include powder, a paste, and a liquid. The above-mentioned states can be appropriately adjusted according to a production method of the composition containing black ginger extract. Moreover, the powder thereof may be further shaped into a solid bulk, granules, or cubes.

### —Production Method—

A production method of a paste or liquid of the composition containing black ginger extract is not particularly limited, and may be appropriately selected depending on the intended purpose. For example, the black ginger extract, the cyclodextrin, and optionally the above-mentioned other ingredients are added and mixed together, and the resultant mixture is vacuum-concentrated by means of a device, such as a rotary evaporator, to thereby produce the paste or liquid of the composition containing black ginger extract.

A production method of powder of the composition containing black ginger extract is not particularly limited, and may be appropriately selected depending on the intended purpose. Examples thereof include a method where the black ginger extract, the cyclodextrin, and optionally the above-mentioned other ingredients are added and mixed together, and the mixture is vacuum-concentrated by a device, such as a rotary evaporator, followed by drying the concentrated product by means of a known dryer, such as a vacuum concentrator, a spray dryer, a hot air dryer, and a freeze dryer. Among the above-listed examples, a method for drying by means of a freeze dryer is preferable.

When a temperature is elevated for drying, the heating conditions are not particularly limited, and may be appropriately selected depending on the intended purpose. The heating is preferably performed for about 16 hours to about 48 hours at about 40° C. to about 70° C.

After drying the composition containing black ginger extract, the dried composition is pulverized by a known pulverization method, for example, using a motor or an impact pulverizer, to thereby obtain powder of the composition containing black ginger extract. Moreover, the powder is optionally sieved, to thereby obtain powder of the composition containing black ginger extract having a uniform particle size.

### —Use—

Since the composition containing black ginger extract has the improved taste of the black ginger extract and the improved solubility to water and an acid solution, the composition containing black ginger extract can be widely used as cosmetic products, beverages and food, medical and pharmaceutical products, regardless of fields. Moreover, the composition containing black ginger extract is suitably used for the below-described composition for oral administration of the present invention.

The composition containing black ginger extract of the present invention is suitably used for humans. A long as the effect of the composition containing black ginger extract is exhibited, the composition containing black ginger extract may be also used for animals other than humans (e.g., mice, rats, hamsters, dogs, cats, cows, pigs, and monkeys).

### (Composition for Oral Administration)

The composition for oral administration of the present invention includes the composition containing black ginger extract of the present invention, and may further include other ingredients according to the necessity.

Since the composition for oral administration of the present invention includes the composition containing black ginger extract, owing to the effects of the black ginger extract that is an active ingredient of the composition containing black ginger extract, the composition for oral administration can be used for nutritional enhancement, such as increasing energy, enhancing sexual performance, improving symptoms of stomach or bowel illness, and improving pain, or for anti-oxidation, anti-aging, anti-inflammatory, hair growth, anti-obesity, skin-brightening, etc.

The composition for oral administration is a composition that rarely harms health of any humans, and can be taken though oral administration or gastrointestinal administration during normal social activities. The composition for oral administration is not something classified as a category, such as food, a medical and pharmaceutical product, or a quasi-drug, according to any law or regulations. Accordingly, the composition for oral administration means a wide range of beverages and food for oral administration, including general food, health food (functional beverages and food), health-promoting food (specified health food, food with nutrient function claims, and food with functional claims), quasi drugs, medical and pharmaceutical products, etc.

A type of the composition for oral administration is not particularly limited, and may be appropriately selected depending on the intended purpose. Examples thereof include: drinks, such as tea, refreshing drinks, carbonated drinks, energy drinks, fruit drinks, lactic acid bacteria drinks, alcoholic drinks, coffee, and coffee-containing soft drinks (including concentrated liquids and powder for preparing any of the above-listed drinks); frozen desserts such as ice cream, ice sherbet, and shaved ice; noodles, such as buckwheat noodles, wheat noodles, vermicelli, Chinese dumpling wrappers, pork dumpling wrappers, Chinese noodles, and instant noodles; sweets and snacks, such as boiled sweets, candies, chewing gum, chocolate, tableted sweets, snacks, biscuits, jelly, jam, cream, backed confectionery, and bread; marine products, such as crab, salmon, Japanese littleneck clam, tuna, sardine, prawns and shrimps, bonito, mackerel, whale, oyster, saury, squid, bloody clam, scallop, abalone, sea chestnut, salmon caviar, and Sulculus diversicolor supertexta; marine/livestock processed food such as a steamed fish paste, ham, and sausages; dairy products, such as processed milk, and fermented milk; oils and fats or processed foods thereof, such as salad oil, Tempura oil, margarine, mayonnaise, shortening, whipping cream and dressing; seasonings, such as source, and dipping source; packed ready-made meals, such as curry, stew, Oyako-don (a bowl of rice topped with cooked chicken and eggs), rice porridge, Zosui(rice in soup), Chuka-don (a bowl of rice topped with a chop-suey stir fry), Katsu-don (a rice bowl with pork cutlets), Ten-don (a bowl of rice topped with tempura), Una-don (a bowl of rice topped with grilled eel dipped in sweat source), hayashi rice (hashed beef with rice), Oden (a dish containing several ingredients cooked in broth, such as boiled eggs and radish), mapo tofu, Gyu-don (a beef rice bowl), meat sauce, egg soup, rice omelet, Chinese dumplings, pork dumplings, hamburger steak and meat balls; ready-made food, such as salad, and pick healthy, cosmetic, and dietary supplemental foods in various forms; pharmaceutical drugs and quasi drugs such as tablets, granules, capsules, drinkable preparations, lozenges, mouthwash; and oral fresheners used inside a mouth, such as mouth freshener, and oral deodorant, and tooth paste. Among the above-listed examples, the type of the composition for oral administration is preferably a drink.

### <Composition Containing Black Ginger Extract>

An amount of the composition containing black ginger extract in the composition for oral administration is nor particularly limited, and may be appropriately adjusted considering intended use, symptoms, and sex of a person to whom the composition is to be administered.

When a type of the composition for oral administration is a drink, the amount of the composition containing black ginger extract in the drink is not particularly limited, and may be appropriately selected depending on the intended purpose, but the amount thereof is preferably 0.01% by mass to 50% by mass relative to the total amount of the composition for oral administration. Depending on cases, the drink including greater than 50% by mass of the composition containing black ginger extract may be used. Moreover, an effective result of the black ginger extract can be obtained with intake of 75 mg to 300 mg per day.

### <Other Ingredients>

Other ingredients in the composition for oral administration are not particularly limited, and may be appropriately selected from auxiliary materials, additives, or other ingredient typically used for production of a composition for oral administration. Examples thereof include various additives, such as acid pH adjusters, glucose, fructose, sucrose, maltose, sorbitol, stevioside, rubusoside, corn syrup, dl-α-tocopherol, sodium erythorbate, glycerin, propylene glycol, fatty acid ester, gum arabic, carrageenan, casein, gelatin, pectin, agar, vitamins B, nicotinic-acid amide, calcium pantothenate, amino acids, calcium salts, dyes, perfumes, preservatives, solvents, stabilizers, and antioxidants. The above-listed examples may be used alone or in combination.

The amount of the above-mentioned other ingredients in the composition for oral administration is not particularly limited, and may be appropriately selected depending on the intended purpose, as long as the effect of the present invention is not adversely affected.

The acid pH adjuster is not particularly limited and may be appropriately selected from pH adjusters known in the art depending on the intended purpose. Examples thereof include phosphoric acid, ascorbic acid, citric acid, tartaric acid, malic acid, lactic acid, phytic acid, gluconic acid, succinic acid, and fumaric acid. The above-listed examples may be used alone, or in combination.

An amount of the acid pH adjuster is not particularly limited, and may be appropriately selected depending on the intended pH.

### EXAMPLES

The present invention will be concretely described through Preparation Examples, Examples, Comparative Examples, and Test Examples, but Preparation Examples, Examples, and Test Examples shall not be construed as limiting the scope of the present disclosure.

### Preparation Example 1: Preparation of Black Ginger Extract

To 5 kg of slices (dried) of a rhizome of black ginger, 50 L of a 60% by volume ethanol aqueous solution was added. The resultant mixture was subjected to heat reflux extraction for 1 hour at 70° C. with gently stirring, to obtain heat reflection extract. Subsequently, the heat reflection extract was filtered with a sieve of 200-mesh (available from IIDA SEISAKUSHO) to remove insoluble substances, to thereby obtain a filtrate. The obtained filtrate was concentrated by means of a rotary evaporator RE-10E-100 (available from SIBATA SCIENTIFIC TECHNOLOGY LTD.) at 60° C. under reduced pressure, followed by freeze-drying the concentrated product. As a result, 420 g of black ginger extract (powder) was obtained.

The obtained black ginger extract was weighed and collected by 100 g. To the collected black ginger extract, 8 L of a 40% by volume ethanol aqueous solution was added. The resultant was stirred to dissolve the black ginger extract, to thereby obtain a black ginger extract solution (may be referred to as an “unpurified extract” hereinafter).

### Preparation Example 2: Preparation of Purified Liquid A and Purified Liquid B

To a 2 L column chromatography system using a synthetic adsorbent (DIAION HP20, available from Mitsubishi Chemical Corporation), 8 L of the unpurified extract obtained in Preparation Example 1 (excluding 100 g of black ginger extract) was fed at 4 L/h, followed by feeding 6 L of a 40% by volume ethanol aqueous solution at 4 L/h, to elute. Fractions were collected by 2 L per fraction, to thereby obtain 7 fractions of the unadsorbed component (the permeated unpurified extract and the 40% by volume ethanol eluate).

Subsequently, 16 L of a 75% by volume ethanol aqueous solution was fed to the column chromatography system at 6 L/h to elute the adsorbed component. Fractions were collected by 2 L per fraction, to thereby obtain 8 fractions of the 75% by volume ethanol eluate. Hereinafter, the 8 fractions may be referred to as “75% by volume ethanol eluate (1).” “75% by volume ethanol eluate (2),” “75% by volume ethanol eluate (3),” “75% by volume ethanol eluate (4).” “75% by volume ethanol eluate (5),” “75% by volume ethanol eluate (6),” “75% by volume ethanol eluate (7),” and “75% by volume ethanol eluate (8),” respectively, in the order of the elution occurred with the 75% by volume ethanol aqueous solution.

Subsequently, 10 L of a 90% by volume ethanol aqueous solution was fed to the column chromatography system at 6 L/h to elute the adsorbed component. Fractions were collected by 2 L per fraction, to thereby obtain 5 fractions of the 90% by volume ethanol eluate. Hereinafter, the 5 fractions may be referred to as “90% by volume ethanol eluate (1),” “90% by volume ethanol eluate (2),” “90% by volume ethanol eluate (3),” “90% by volume ethanol eluate (4).” and “90% by volume ethanol eluate (5),” respectively, in the order of the elution occurred with the 90% by volume ethanol aqueous solution.

The obtained 8 fractions of the 75% by volume ethanol eluates (1) to (8) and the obtained 5 fractions of the 90% by volume ethanol eluates (1) to (5) were each fed to a high-performance liquid chromatography (HPLC) system under the following analysis conditions, to thereby quantify a total amount of 6 polymethoxyflavones (6PMF), i.e., 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, and 3,5,7,4′-tetramethoxyflavone in each eluate, and an amount of 5-hydroxy-7-methoxyflavone(5-HMF) in each eluate.

### <Analysis Conditions>

### [Preparation of Sample for Analysis]

In a 50 mL volumetric flask, each faction of 75% by volume ethanol eluates (1) to (8) and 90% by volume ethanol eluates (1) to (5) was collected by 1 mL. To the flask, a 60% by volume methanol aqueous solution was added to increase a total volume to 50 mL. Subsequently, the resultant mixture was filtered with a 0.45 μm-membrane filter, to thereby prepare an analysis sample for HPLC.

### [Preparation of Reference Liquid for Calibration Curve]

Reference materials of 6 polymethoxyflavones presented as S1 to S6 below were each accurately weighed and collected by 2 mg, and the collected material was dissolved in 1 mL of dimethyl sulfoxide. Tb the resultant solution, 100% by volume methanol was added to increase the total volume to 20 mL. The resultant was serial-diluted with a 60% by volume methanol aqueous solution to prepare a solution. The resultant solution was filtered with a membrane filter (pore diameter: 0.45 μm), to thereby prepare a reference liquid of each of 6 polymethoxyflavones for a calibration curve.

Moreover, a reference material of 5-hydroxy-7-methoxyflavone presented as S7 below was accurately weighed and collected by 2 g. The collected material was dissolved in 1 mL of dimethyl sulfoxide. To the resultant solution, 100% by volume methanol was added to increase the total volume to 20 mL. The resultant was serial-diluted with a 60% by volume methanol aqueous solution to prepare a solution. The resultant solution was filtered with a membrane filter (pore diameter: 0.45 μm), to thereby prepare a reference liquid of 5-hydroxy-7-methoxyflavone for a calibration curve.

S1: 5,7,3′,4′-tetramethoxyflavone (available from INDOFINE Chemical Company, Inc.)
S2: 3,5,7,3′,4′-pentamethoxyflavone (available from Tokiwa Phytochemical Co., Ltd.)
S3: 5,7-dimethoxyflavone (available from INDOFINE Chemical Company, Inc.)
S4: 5,7,4′-trimethoxyflavone (available from INDOFINE Chemical Company, Inc.)
S5: 3,5,7-trimethoxyflavone (available from Tokiwa Phytochemical Co., Ltd.)
S6: 3,5,7,4′-tetramethoxyflavone (available from Tokiwa Phytochemical Co., Ltd.)
S7: 5-hydroxy-7-methoxyflavone (available from INDOFINE Chemical Company, Inc.)

### [HPLC Analysis Conditions]

Analysis device: high-performance liquid chromatography (available from Shimadzu Corporation)
Column: Ascentis (registered trademark) Express C18 (length: 10 cm, inner diameter: 4.6 mm, particle diameter: 2.7 μm, available from Sigma-Aldrich Japan)
Detector: photo diode array (PDA) (available from Shimadzu Corporation)
Detecting wavelength: 265 nm (wavelength of PDA: 200 nm to 440 nm)
Column temperature: 40° C.
Mobile phase: a (0.1% by volume)trifluoroacetic acid-containing (60% by volume) methanol aqueous solution
Analysis time: 60 minutes
Flow rate: 0.6 mL/min
Sample injection amount for analysis: 10 μL

A combination of the obtained 6 fractions of 75% by volume ethanol eluates (1) to (6) was provided as “purified liquid A” (9.9 kg).

Moreover, a combination the obtained 2 fractions of 75% by volume ethanol eluates (7) and (8), and the obtained 5 fractions of 90% by volume ethanol eluates (1) to (5) were provided as “purified liquid B” (11.7 kg).

The total amount of 6PMF in the purified liquid A was 2.44 mg/g and the amount of 5-HMF in the purified liquid A was 0.003 mg/g. The total amount of 6PMF and the amount of 5-HMF in the purified liquid A were calculated from the amounts of the 6 polymethoxyflavones (6PMF) and 5-hydroxy-7-methoxyflavone(5-HMF) in each elute quantified under the above-described analysis conditions. Moreover, the solid content in the purified liquid A1 was 2.85 mg/g.

Moreover, the total amount of 6PMF in the purified liquid B was 0.09 mg/g and the amount of 5-HMF in the purified liquid B was 0.09 mg/g. The total amount of 6PMF and the amount of 5-HMF in the purified liquid B were calculated from the amounts of the 6 polymethoxyflavones (6PMF) and 5-hydroxy-7-methoxyflavone (5-HMF) in each eluate quantified under the above-described analysis conditions. Moreover, the solid content in the purified liquid B was 0.47 mg/g.

The solid content in the purified liquid A was a mass of the solids per gram of the purified liquid A, and the solids were obtained by freeze-drying the purified liquid A. Similarly, moreover, the solid content in the purified liquid B was a mass of the solids per gram of the purified liquid B, and the solids were obtained by freeze-drying the purified liquid B.

### Example 1

In 100 mL of water, 3.7 g of γ-cyclodextrin (CAVAMAX (registered trademark) W8 Food, available from CycloChem Co., Ltd.) was dissolved to prepare a solution. To the γ-cyclodextrin solution, 192 g (547.5 mg on solid bases) of the purified liquid A obtained in Preparation Example 2 was added and mixed together, to thereby obtain a mixed solution. Subsequently, the mixed solution was subjected to vacuum concentration by means of a rotary evaporator N-1100 (available from TOKYO RIKAKIKAI CO., LTD.). The obtained concentrated liquid was freeze-dried, and the dried product was pulverized by a mortar to turn into powder, to thereby obtain 4.3 g of a composition containing black ginger extract of Example 1.

### Example 2

In 100 mL of water, 3.8 g of γ-cyclodextrin (CAVAMAX (registered trademark) W8 Food, available from CycloChem Co., Ltd.) was dissolved to prepare a solution. Moreover, 192 g (547.5 mg on solid basis) of the purified liquid A obtained in Preparation Example 2, and 8 g (3.8 mg on solid basis) of the purified liquid B obtained in Preparation Example 2 were mixed together, to thereby obtain a mixture of the purified liquids. To the γ-cyclodextrin solution, the mixture of the purified liquids was added and mixed together to thereby obtain a mixed solution. Subsequently, the mixed solution was subjected to vacuum concentration by means of a rotary evaporator N-1100 (available from TOKYO RIKAKIKAI CO., LTD.). The obtained concentrated liquid was freeze-dried, and the dried product was pulverized by a mortar to turn into powder, to thereby obtain 4.3 g of a composition containing black ginger extract of Example 2.

### Example 3

In 100 mL of water, 3.5 g of γ-cyclodextrin (CAVAMAX (registered trademark) W8 Food, available from CycloChem Co., Ltd.) was dissolved to prepare a solution. Moreover, 176 g (501.9 mg on solid basis) of the purified liquid A obtained in Preparation Example 2, and 24 g (11.4 mg on solid basis) of the purified liquid B obtained in Preparation Example 2 were mixed together, to thereby obtain a mixture of the purified liquids. To the γ-cyclodextrin solution, the mixture of the purified liquids was added and mixed together to thereby obtain a mixed solution. Subsequently, the mixed solution was subjected to vacuum concentration by means of a rotary evaporator N-1100 (available from TOKYO RIKAKIKAI CO., LTD.). The obtained concentrated liquid was freeze-dried, and the dried product was pulverized by a mortar to turn into powder, to thereby obtain 4.0 g of a composition containing black ginger extract of Example 3.

### Example 4

In 100 mL of water, 2.9 g of γ-cyclodextrin (CAVAMAX (registered trademark) W8 Food, available from CycloChem Co., Ltd.) was dissolved to prepare a solution. Moreover, 148 g (422.1 mg on solid basis) of the purified liquid A obtained in Preparation Example 2, and 52 g (24.6 mg on solid basis) of the purified liquid B obtained in Preparation Example 2 were mixed together, to thereby obtain a mixture of the purified liquids. To the γ-cyclodextrin solution, the mixture of the purified liquids was added and mixed together to thereby obtain a mixed solution. Subsequently, the mixed solution was subjected to vacuum concentration by means of a rotary evaporator N-1100 (available from TOKYO RIKAKIKAI CO., LTD.). The obtained concentrated liquid was freeze-dried, and the dried product was pulverized by a mortar to turn into powder, to thereby obtain 3.4 g of a composition containing black ginger extract of Example 4.

### Example 5

In 100 mL of water, 2.5 g of γ-cyclodextrin (CAVAMAX (registered trademark) W8 Food, available from CycloChem Co., Ltd.) was dissolved to prepare a solution. Moreover, 124 g (353.6 mg on solid basis) of the purified liquid A obtained in Preparation Example 2, and 76 g (36.0 mg on solid basis) of the purified liquid B obtained in Preparation Example 2 were mixed together, to thereby obtain a mixture of the purified liquids. To the γ-cyclodextrin solution, the mixture of the purified liquids was added and mixed together to thereby obtain a mixed solution. Subsequently, the mixed solution was subjected to vacuum concentration by means of a rotary evaporator N-1100 (available from TOKYO RIKAKIKAI CO., LTD.). The obtained concentrated liquid was freeze-dried, and the dried product was pulverized by a mortar to turn into powder, to thereby obtain 2.9 g of a composition containing black ginger extract of Example 5.

### Example 6

In 100 mL of water, 3.2 g of γ-cyclodextrin (CAVAMAX (registered trademark) W8 Food, available from CycloChem Co., Ltd.) was dissolved to prepare a solution. Moreover, 160 g (456.3 mg on solid basis) of the purified liquid A obtained in Preparation Example 2, and 136 g (64.4 mg on solid basis) of the purified liquid B obtained in Preparation Example 2 were mixed together, to thereby obtain a mixture of the purified liquids. To the γ-cyclodextrin solution, the mixture of the purified liquids was added and mixed together to thereby obtain a mixed solution. Subsequently, the mixed solution was subjected to vacuum concentration by means of a rotary evaporator N-1100 (available from TOKYO RIKAKIKAI CO., LTD.). The obtained concentrated liquid was freeze-dried, and the dried product was pulverized by a mortar to turn into powder, to thereby obtain 3.7 g of a composition containing black ginger extract of Example 6.

### Comparative Example 1

In 100 mL of water, 3.4 g of γ-cyclodextrin (CAVAMAX (registered trademark) W8 Food, available from CycloChem Co., Ltd.) was dissolved to prepare a solution. Moreover, 168 g (479.1 mg on solid basis) of the purified liquid A obtained in Preparation Example 2, and 232 g (109.9 mg on solid basis) of the purified liquid B obtained in Preparation Example 2 were mixed together, to thereby obtain a mixture of the purified liquids. To the γ-cyclodextrin solution, the mixture of the purified liquids was added and mixed together to thereby obtain a mixed solution. Subsequently, the mixed solution was subjected to vacuum concentration by means of a rotary evaporator N-1100 (available from TOKYO RIKAKIKAI CO., LTD.). The obtained concentrated liquid was freeze-dried, and the dried product was pulverized by a mortar to turn into powder, to thereby obtain 4.0 g of a composition containing black ginger extract of Comparative Example 1.

### Comparative Example 2

In 100 mL of water, 2.4 g of γ-cyclodextrin (CAVAMAX (registered trademark) W8 Food, available from CycloChem Co., Ltd.) was dissolved to prepare a solution. Moreover, 112 g (319.4 mg on solid basis) of the purified liquid A obtained in Preparation Example 2, and 304 g (144.0 mg on solid basis) of the purified liquid B obtained in Preparation Example 2 were mixed together, to thereby obtain a mixture of the purified liquids. To the γ-cyclodextrin solution, the mixture of the purified liquids was added and mixed together to thereby obtain a mixed solution. Subsequently, the mixed solution was subjected to vacuum concentration by means of a rotary evaporator N-1100 (available from TOKYO RIKAKIKAI CO., LTD.). The obtained concentrated liquid was freeze-dried, and the dried product was pulverized by a mortar to turn into powder, to thereby obtain 2.9 g of a composition containing black ginger extract of Comparative Example 2.

### Comparative Example 3

In 100 mL of water, 3.6 g of γ-cyclodextrin (CAVAMAX (registered trademark) W8 Food, available from CycloChem Co., Ltd.) was dissolved to prepare a solution. After dissolving 1.5 g of the black ginger extract obtained in Preparation Example 1 in 100 mL of 60% by volume ethanol, the resultant solution was added to the γ-cyclodextrin solution, to thereby obtain a mixed solution. Subsequently, the mixed solution was subjected to vacuum concentration by means of a rotary evaporator N-1100 (available from TOKYO RIKAKIKAI CO., LTD.). The obtained concentrated liquid was freeze-dried, and the dried product was pulverized by a mortar to turn into powder, to thereby obtain 5.1 g of a composition containing black ginger extract of Comparative Example 3.

### Examples 7, 13, and 19

Compositions each containing black ginger extract of Examples 7, 13, and 19 were prepared in the same manner as the preparation method of the composition containing black ginger extract of Example 1, except that the amount of γ-cyclodextrin was changed to the amount presented in Tables 2 to 4.

### Examples 8, 14, and 20

Compositions each containing black ginger extract of Examples 8, 14, and 20 were prepared in the same manner as the preparation method of the composition containing black ginger extract of Example 2, except that the amount of γ-cyclodextrin was changed to the amount presented in Tables 2 to 4.

### Examples 9, 15, and 21

Compositions each containing black ginger extract of Examples 9, 15, and 21 were prepared in the same manner as the preparation method of the composition containing black ginger extract of Example 3, except that the amount of γ-cyclodextrin was changed to the amount presented in Tables 2 to 4.

### Examples 10, 16, and 22

Compositions each containing black ginger extract of Examples 10, 16, and 22 were prepared in the same manner as the preparation method of the composition containing black ginger extract of Example 4, except that the amount of γ-cyclodextrin was changed to the amount presented in Tables 2 to 4.

### Examples 11, 17, and 23

Compositions each containing black ginger extract of Examples 11, 17, and 23 were prepared in the same manner as the preparation method of the composition containing black ginger extract of Example 5, except that the amount of γ-cyclodextrin was changed to the amount presented in Tables 2 to 4.

### Examples 12, 18, and 24

Compositions each containing black ginger extract of Examples 12, 18, and 24 were prepared in the same manner as the preparation method of the composition containing black ginger extract of Example 6, except that the amount of γ-cyclodextrin was changed to the amount presented in Tables 2 to 4.

### Comparative Examples 4, 7, and 10

Compositions each containing black ginger extract of Comparative Examples 4, 7, and 10 were prepared in the same manner as the preparation method of the composition containing black ginger extract of Comparative Example 1, except that the amount of γ-cyclodextrin was changed to the amount presented in Tables 2 to 4.

### Comparative Examples 5, 8, and 11

Compositions each containing black ginger extract of Comparative Examples 5, 8, and 11 were prepared in the same manner as the preparation method of the composition containing black ginger extract of Comparative Example 2, except that the amount of γ-cyclodextrin was changed to the amount presented in Tables 2 to 4.

### Comparative Examples 6, 9, and 12

Compositions each containing black ginger extract of Comparative Examples 6, 9, and 12 were prepared in the same manner as the preparation method of the composition containing black ginger extract of Comparative Example 3, except that the amount of γ-cyclodextrin was changed to the amount presented in Tables 2 to 4.

### Test Example 1

### —Measurement of Amounts of 6PMF and 5-HMF—

Amounts (total amount) of 6PMF and an amount of 5-HMF in the composition containing black ginger extract of each of Examples 1 to 24 and Comparative Examples 1 to 12 were quantified in the same analysis method of Preparation Example 2, except that [Preparation of sample for analysis] was changed to the following method. Based on the quantified values, the amount (mass) of 5-HMF in the composition containing black ginger extract relative to 100 parts by mass of the total amount of 6PMF in the composition containing black ginger extract was calculated. The results are presented in Tables 1 to 4 below.

### [Preparation of Samples for Analysis]

The composition containing black ginger extract (powder) of each of Examples 1 to 24 and Comparative Examples 1 to 12 was weighed and collected by 10 mg, and the collected composition was dissolved in 60% by mass methanol. Moreover, 60% by volume methanol was added to the resultant solution to increase. the total volume to 50 mL. Subsequently, the resultant was filtered with a membrane filter (pore diameter: 0.45 μm), to thereby prepare an analysis sample for HPLC.

### Test Example 2

### —Measurement of Absorbance—

Distilled water was added to the composition containing black ginger extract (powder) of each of Examples 1 to 24 and Comparative Examples 1 to 12 to prepare a composition containing black ginger extract aqueous solution, in which a total amount of 6PMF was 0.006 mg/mL. The prepared aqueous solution was provided as a test sample.

A quartz cell having a light path length was charged with the test sample of each of Examples 1 to 24 and Comparative Examples 1 to 12, and absorbance was measured by means of an ultraviolet-visible spectrophotometer (model number: UV-1800, available from Shimadzu Corporation) by scanning absorbance from the wavelength of 200 nm to 450 nm at intervals of 2 nm or less, to thereby obtain an ultraviolet (UV) absorption spectrum.

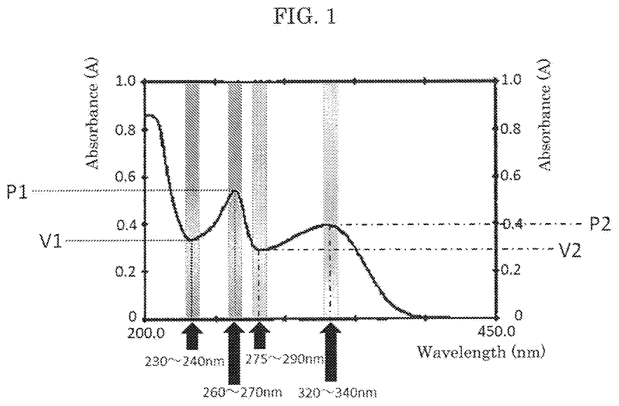

The ultraviolet (UV) absorption spectrum of Example 1 is presented in FIG. 1. In Example 1, the minimum value V1 of the absorbance in the wavelength range of from 230 nm to 240 nm, the maximum value P1 of the absorbance in the wavelength range of from 260 nm to 270 nm, the minimum value V2 in the wavelength range of from 275 nm to 290 nm, and the maximum value P2 in the wavelength range of from 320 nm to 340 nm were confirmed from the ultraviolet (UV) absorption spectrum of FIG. 1. From the confirmed values of absorbance, the absorbance ratio (P1/V1) and the absorbance ratio (P2/V2) were calculated. V1, P1, V2, and P2 were values that were background-corrected using distilled water as a control sample.

Although it is not illustrated in FIG. 1, in Examples 2 to 24 and Comparative Examples 1 to 12, the absorbance ratio (P1/V1) and the absorbance ratio (P2/V2) were calculated in the same manner. The results are presented in Tables 1 to 4 below.

### Test Example 3

### —Measurement of Transmittance—

Distilled water or a 0.1% by mass citric acid solution was added to the composition containing black ginger extract (powder) of each of Examples 1 to 24 and Comparative Examples 1 to 12 to prepare a composition containing black ginger extract aqueous solution, in which a total amount of 6PMF was 0.05 mg/mL. The prepared aqueous solution was processed with ultrasonic waves for 5 minutes, heated for 20 minutes at 80° C., followed by cooling to room temperature, to thereby prepare a test sample.

A quartz cell having a light path length of 10 mm was charged with the test sample of each of Examples 1 to 24 and Comparative Examples 1 to 12, and transmittance (T %) to light having a wavelength of 660 nm was measured by means of an ultraviolet-visible spectrophotometer (model number: UV-1800, available from Shimadzu Corporation). The transmittance of the test sample of each of Examples 1 to 24 and Comparative Examples 1 to 12 to light was calculated with using distilled water or a 0.1% by mass citric acid solution as a control sample, and determining transmittance (T %) of distilled water to light having a wavelength of 660 nm as 100%. The results are presented in Tables 1 to 4 below.

### Test Example 4

### —Taste Test by Taste Sensor—

Among the compositions containing black ginger extract (powder) of Examples 1 to 24 and Comparative Examples 1 to 12, a taste test was performed on some of the compositions containing black ginger extract of Examples and Comparative Examples by means of a taste sensor. In Tables 1 to 4 below, Examples in which the taste test by the taste sensor was not performed were marked as “N.D.”

Specifically, a 20% by volume ethanol solution including 10 mM potassium chloride was added to the composition containing black ginger extract (powder) of each of Examples and Comparative Examples, to prepare a solution in which the total amount of 6PMF was 0.24 mg/mL. The prepared solution was provided as a sample for analysis.

Next, bitterness analysis of the analysis sample was performed by means of a taste sensor (taste sensing system TS-5000Z, available from Intelligent Sensor Technology, Inc.) with a bitterness sensor (C00, available from Intelligent Sensor Technology, Inc.) and a saltiness sensor (CT0, available from Intelligent Sensor Technology, Inc.).

The obtained output data of the sensor was converted into a numerical value indicating bitterness by an analysis application (using data for ECE food evaluation) installed in the taste sensing system (TS-5000Z), using the data of the composition containing black ginger extract (Comparative Example) prepared using the black ginger extract obtained in Preparation Example 1 as a reference (0). Specifically, the following reference (0) was used in each of Examples and Comparative Examples.

Examples 1, 3, and 6, and Comparative Examples 1 and 2 used Comparative Example 3 as a reference (0).
Examples 7, 9, and 12, and Comparative Examples 4 and 5 used Comparative Example 6 as a reference (0).
Examples 13, 15, and 18, and Comparative Examples 7 and 8 used Comparative Example 9 as a reference (0).
Examples 19, 21, and 24, and Comparative Examples 10 and 11 used Comparative Example 12 as a reference (0).

The results are presented in Tables 1 to 4 below. The numerical value of the bitterness obtained by the taste sensor indicated as follows. When the numerical value was positive relative to the reference (0), bitterness was strong. When the numerical value was negative relative to reference (0), bitterness was weak.

### Test Example 5

### —Taste Test by Sensory Evaluation—

Among the compositions containing black ginger extract (powder) of Examples 1 to 24 and Comparative Examples 1 to 12, a taste test was performed on some of the compositions containing black ginger extract of Examples and Comparative Examples by a sensory evaluation. In Tables 1 to 4 below, Examples in which the taste test by the sensory evaluation was not performed were marked as “N.D.”

Specifically, water was added to the composition containing black ginger extract (powder) of each of Examples and Comparative Examples to prepare a composition containing black ginger extract aqueous solution in which the total amount of 6 PMF was 0.03 mg/mL. The obtained composition containing black ginger extract aqueous solution was used as a sample.

The sample was orally administered to a panel of 10 specialists who were all adults (5 makes and 5 females), and the specialists evaluated the bitterness of the sample based on the following evaluation criteria. The average value of the evaluation results from all of the specialists is presented in Tables 1 to 4 below.

The sample was provided in a manner of a double blind trial, and the evaluation was performed while the panel of specialists were separated from one another. Moreover, the sample of the composition containing black ginger extract (Comparative Example) prepared using the black ginger extract prepared in Preparation Example 1 was used as a reference sample of “Evaluation criteria 1: very bitter sample.” Specifically, the following reference sample was used for each of Examples and Comparative Examples.

Examples 1, 3, 5, and 6, and Comparative Example 1 used Comparative Example 3 as a reference sample.
Examples 7, 9, 11, and 12, and Comparative Example 4 used Comparative Example 6 as a reference sample.
Examples 13, 15, 17, and 18, and Comparative Example 7 used Comparative Example 9 as a reference sample.
Examples 19, 21, 23, and 24, and Comparative Example 10 used Comparative Example 12 as a reference sample.

### —Evaluation Criteria of Bitterness—

5: Not bitter
4: Very slightly bitter
3: Slightly bitter
2: Notably bitter
1: Very bitter (reference sample)

TABLE 1

| | | Example | | | | | | Comparative Example | | |
|---|---|---|---|---|---|---|---|---|---|---|
| | | 1 | 2 | 3 | 4 | 5 | 6 | 1 | 2 | 3 |
| Composition | Purified liquid | 192 | 192 | 176 | 148 | 124 | 160 | 168 | 112 | — |
| | A (g) | | | | | | | | | |
| | Purified liquid | — | 8 | 24 | 52 | 76 | 136 | 232 | 304 | — |
| | B (g) | | | | | | | | | |
| | Black ginger | — | — | — | — | — | — | — | — | 1.5 |
| | extract (g) | | | | | | | | | |
| | γ-cyclodextrin | 3.7 | 3.8 | 3.5 | 2.9 | 2.5 | 3.2 | 3.4 | 2.4 | 3.6 |
| | (g) | | | | | | | | | |
| Composition containing black | | 4.3 | 4.3 | 4.0 | 3.4 | 2.9 | 3.7 | 4.0 | 2.9 | 5.1 |
| ginger extract (dry mass) (g) | | | | | | | | | | |
| Total amount of 6PMF (mg/g) | | 109.07 | 108.99 | 108.82 | 108.45 | 108.00 | 107.60 | 106.75 | 104.81 | 88.24 |
| Amount of 5-HMF (mg/g) | | 0.13 | 0.31 | 0.71 | 1.60 | 2.65 | 3.58 | 5.59 | 10.18 | 4.71 |
| Amount of 5-HMF relative to 100 | | 0.1 | 0.3 | 0.7 | 1.5 | 2.5 | 3.3 | 5.2 | 9.7 | 5.3 |
| parts by mass of total amount | | | | | | | | | | |
| of 6PMF (parts by mass) | | | | | | | | | | |
| Amount of γ-cyclodextrin | | 8.0 | 8.0 | 8.0 | 8.0 | 8.0 | 8.0 | 8.0 | 8.0 | 8.0 |
| relative to 1 part by mass of | | | | | | | | | | |
| total amount of 6PMF (parts | | | | | | | | | | |
| by mass) | | | | | | | | | | |
| P1/V1 (absorbance ratio) | | 1.47 | 1.51 | 1.52 | 1.52 | 1.53 | 1.53 | 1.54 | 1.56 | 1.37 |
| P2/V2 (absorbance ratio) | | 1.52 | 1.47 | 1.46 | 1.45 | 1.44 | 1.43 | 1.41 | 1.37 | 1.31 |
| Evaluation | Transmittance | 99.8 | 99.7 | 99.6 | 99.5 | 99.0 | 97.2 | 86.4 | 71.0 | 93.2 |
| results | of aqueous | | | | | | | | | |
| | solution (T %) | | | | | | | | | |
| | Transmittance | 99.9 | 99.7 | 99.6 | 99.4 | 98.6 | 95.7 | 86.6 | 65.2 | 92 |
| | of acid solution | | | | | | | | | |
| | (T %) | | | | | | | | | |
| | Bitterness | −3.7 | N.D. | −3.6 | N.D. | N.D. | −3.5 | −3.0 | −3.1 | 0 (ref.) |
| | (Taste sensor) | | | | | | | | | |
| | Bitterness | 4.2 | N.D. | 4.1 | N.D. | 3.8 | 3.3 | 2.4 | N.D. | 1.0 (ref.) |
| | (Sensory | | | | | | | | | |
| | evaluation) | | | | | | | | | |

TABLE 2

| | | Example | | | | | | Comparative Example | | |
|---|---|---|---|---|---|---|---|---|---|---|
| | | 7 | 8 | 9 | 10 | 11 | 12 | 4 | 5 | 6 |
| Composition | Purified liquid | 192 | 192 | 176 | 148 | 124 | 160 | 168 | 112 | — |
| | A (g) | | | | | | | | | |
| | Purified liquid | — | 8 | 24 | 52 | 76 | 136 | 232 | 304 | — |
| | B (g) | | | | | | | | | |
| | Black ginger | — | — | — | — | — | — | — | — | 1.5 |
| | extract (g) | | | | | | | | | |
| | γ-cyclodextrin | 2.8 | 2.8 | 2.6 | 2.2 | 1.9 | 2.4 | 2.6 | 1.8 | 2.7 |
| | (g) | | | | | | | | | |
| Composition containing black | | 3.4 | 3.4 | 3.1 | 2.6 | 2.2 | 2.9 | 3.2 | 2.3 | 4.2 |
| ginger extract (dry mass) (g) | | | | | | | | | | |
| Total amount of 6PMF (mg/g) | | 139.49 | 139.37 | 139.10 | 138.48 | 137.75 | 137.11 | 135.74 | 132.60 | 107.14 |
| Amount of 5-HMF (mg/g) | | 0.17 | 0.40 | 0.90 | 2.04 | 3.38 | 4.56 | 7.10 | 12.88 | 5.71 |
| Amount of 5-HMF relative to 100 | | 0.1 | 0.3 | 0.7 | 1.5 | 2.5 | 3.3 | 5.2 | 9.7 | 5.3 |
| parts by mass of total amount | | | | | | | | | | |
| of 6PMF (parts by mass) | | | | | | | | | | |
| Amount of γ-cyclodextrin | | 6.0 | 6.0 | 6.0 | 6.0 | 6.0 | 6.0 | 6.0 | 6.0 | 6.0 |
| relative to 1 part by mass of | | | | | | | | | | |
| total amount of 6PMF (parts by | | | | | | | | | | |
| mass) | | | | | | | | | | |
| P1/V1 (absorbance ratio) | | 1.47 | 1.51 | 1.52 | 1.52 | 1.53 | 1.53 | 1.54 | 1.56 | 1.37 |
| P2/V2 (absorbance ratio) | | 1.52 | 1.47 | 1.46 | 1.45 | 1.44 | 1.43 | 1.41 | 1.37 | 1.31 |
| Evaluation | Transmittance | 99.8 | 99.7 | 99.5 | 99.2 | 98.8 | 97.8 | 86.2 | 70.2 | 92.2 |
| results | of aqueous | | | | | | | | | |
| | solution (T %) | | | | | | | | | |
| | Transmittance | 99.7 | 99.6 | 99.4 | 98.8 | 98.4 | 96.3 | 85.8 | 68.1 | 91.5 |
| | of acid solution | | | | | | | | | |
| | (T %) | | | | | | | | | |
| | Bitterness | −3.6 | N.D. | −3.6 | N.D. | N.D. | −3.6 | −3.0 | −3.0 | 0 (ref.) |
| | (Taste sensor) | | | | | | | | | |
| | Bitterness | 4.1 | N.D. | 4.1 | N.D. | 3.8 | 3.3 | 2.2 | N.D. | 1.0 (ref.) |
| | (Sensory | | | | | | | | | |
| | evaluation) | | | | | | | | | |

TABLE 3

| | | Example | | | | | | Comparative Example | | |
|---|---|---|---|---|---|---|---|---|---|---|
| | | 13 | 14 | 15 | 16 | 17 | 18 | 7 | 8 | 9 |
| Composition | Purified liquid | 192 | 192 | 176 | 148 | 124 | 160 | 168 | 112 | — |
| | A (g) | | | | | | | | | |
| | Purified liquid | — | 8 | 24 | 52 | 76 | 136 | 232 | 304 | — |
| | B (g) | | | | | | | | | |
| | Black ginger | — | — | — | — | — | — | — | — | 1.5 |
| | extract (g) | | | | | | | | | |
| | γ-cyclodextrin | 2.3 | 2.3 | 2.2 | 1.8 | 1.5 | 2.0 | 2.2 | 1.5 | 2.3 |
| | (g) | | | | | | | | | |
| Composition containing black | | 2.9 | 2.9 | 2.7 | 2.3 | 1.9 | 2.5 | 2.7 | 2.0 | 3.8 |
| ginger extract (dry mass) (g) | | | | | | | | | | |
| Total amount of 6PMF (mg/g) | | 162.11 | 161.94 | 161.57 | 160.74 | 159.76 | 158.90 | 157.95 | 152.87 | 120.00 |
| Amount of 5-HMF (mg/g) | | 0.20 | 0.46 | 1.05 | 2.37 | 3.92 | 5.29 | 8.22 | 14.85 | 6.40 |
| Amount of 5-HMF relative to 100 | | 0.1 | 0.3 | 0.7 | 1.5 | 2.5 | 3.3 | 5.2 | 9.7 | 5.3 |
| parts by mass of total amount | | | | | | | | | | |
| of 6PMF (parts by mass) | | | | | | | | | | |
| Amount of γ-cyclodextrin | | 5.0 | 5.0 | 5.0 | 5.0 | 5.0 | 5.0 | 5.0 | 5.0 | 5.0 |
| relative to 1 part by mass of | | | | | | | | | | |
| total amount of 6PMF (parts | | | | | | | | | | |
| by mass) | | | | | | | | | | |
| P1/V1 (absorbance ratio) | | 1.47 | 1.51 | 1.52 | 1.52 | 1.53 | 1.53 | 1.54 | 1.56 | 1.37 |
| P2/V2 (absorbance ratio) | | 1.52 | 1.47 | 1.46 | 1.45 | 1.44 | 1.43 | 1.41 | 1.37 | 1.31 |
| Evaluation | Transmittance | 99.7 | 99.7 | 99.6 | 99.2 | 98.5 | 95.9 | 85.5 | 68.2 | 91.9 |
| results | of aqueous | | | | | | | | | |
| | solution (T %) | | | | | | | | | |
| | Transmittance | 99.7 | 99.6 | 99.5 | 98.9 | 98.1 | 96.1 | 85.2 | 63.1 | 90.3 |
| | of acid solution | | | | | | | | | |
| | (T %) | | | | | | | | | |
| | Bitterness | −3.7 | N.D. | −3.6 | N.D. | N.D. | −3.6 | −3.1 | −3.1 | 0 (ref.) |
| | (Taste sensor) | | | | | | | | | |
| | Bitterness | 4.1 | N.D. | 4.0 | N.D. | 3.9 | 3.4 | 2.5 | N.D. | 1.0 (ref.) |
| | (Sensory | | | | | | | | | |
| | evaluation) | | | | | | | | | |

TABLE 4

| | | Example | | | | | | Comparative Example | | |
|---|---|---|---|---|---|---|---|---|---|---|
| | | 19 | 20 | 21 | 22 | 23 | 24 | 10 | 11 | 12 |
| Composition | Purified liquid | 192 | 192 | 176 | 148 | 124 | 160 | 168 | 112 | — |
| | A (g) | | | | | | | | | |
| | Purified liquid | — | 8 | 24 | 52 | 76 | 136 | 232 | 301 | — |
| | B (g) | | | | | | | | | |
| | Black ginger | — | — | — | — | — | — | — | — | 1.5 |
| | extract (g) | | | | | | | | | |
| | γ-cyclodextrin | 1.9 | 1.9 | 1.7 | 1.5 | 1.2 | 1.6 | 1.7 | 1.2 | 1.8 |
| | (g) | | | | | | | | | |
| Composition containing black | | 2.4 | 2.4 | 2.2 | 1.9 | 1.6 | 2.1 | 2.3 | 1.7 | 3.3 |
| ginger extract (dry mass) (g) | | | | | | | | | | |
| Total amount of 6PMF (mg/g) | | 193.47 | 193.23 | 192.71 | 191.53 | 190.14 | 188.92 | 186.32 | 180.46 | 136.36 |
| Amount of 5-HMF (mg/g) | | 0.24 | 0.55 | 1.25 | 2.82 | 4.67 | 6.29 | 9.75 | 17.53 | 7.27 |
| Amount of 5-HMF relative to 100 | | 0.1 | 0.3 | 0.7 | 1.5 | 2.5 | 3.3 | 5.2 | 9.7 | 5.3 |
| parts by mass of total amount | | | | | | | | | | |
| of 6PMF (parts by mass) | | | | | | | | | | |
| Amount of γ-cyclodextrin | | 4.0 | 4.0 | 4.0 | 4.0 | 4.0 | 4.0 | 4.0 | 4.0 | 4.0 |
| relative to 1 part by mass of | | | | | | | | | | |
| total amount of 6PMF (parts | | | | | | | | | | |
| by mass) | | | | | | | | | | |
| P1/V1 (absorbance ratio) | | 1.47 | 1.51 | 1.52 | 1.52 | 1.53 | 1.53 | 1.54 | 1.56 | 1.37 |
| P2/V2 (absorbance ratio) | | 1.52 | 1.47 | 1.46 | 1.45 | 1.44 | 1.43 | 1.41 | 1.37 | 1.31 |
| Evaluation | Transmittance | 99.7 | 99.6 | 99.5 | 99.2 | 97.8 | 95.0 | 83.1 | 62.2 | 88.5 |
| results | of aqueous | | | | | | | | | |
| | solution (T %) | | | | | | | | | |
| | Transmittance | 99.6 | 99.5 | 99.4 | 98.8 | 97.4 | 95.0 | 80.2 | 59.6 | 85.9 |
| | of acid solution | | | | | | | | | |
| | (T %) | | | | | | | | | |
| | Bitterness | −3.6 | N.D. | −3.6 | N.D. | N.D. | −3.5 | −3.0 | −2.9 | 0 (ref.) |
| | (Taste sensor) | | | | | | | | | |
| | Bitterness | 4.0 | N.D. | 4.0 | N.D. | 3.9 | 3.3 | 2.2 | N.D. | 1.0 (ref.) |
| | (Sensory | | | | | | | | | |
| | evaluation) | | | | | | | | | |

For example, embodiments of the present invention are as follows.

<1> A composition containing black ginger extract, including:
black ginger extract; and
cyclodextrin,
wherein the composition containing black ginger extract includes
6 polymethoxyflavones including 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, and 3,5,7,4′-tetramethoxyflavone,
when a total amount of the 6 polymethoxyflavones in the composition containing black ginger extract is determined as 100 parts by mass, an amount of 5-hydroxy-7-methoxyflavone in the composition containing black ginger extract is 5 parts by mass or less,
when absorbance of an aqueous solution of the composition containing black ginger extract, in which a total amount of the 6 polymethoxyflavones is adjusted to 0.006 mg/mL, is measured, the absorbance of the aqueous solution satisfies Formula (1) below:

P1/V1≥1.45 Formula(1)

where, in Formula (1), V1 is the minimum value of absorbance in a wavelength range of from 230 nm to 240 nm, and P1 is the maximum value of absorbance in a wavelength range of from 260 nm to 270 nm.

<2> The composition containing black ginger extract according to <1>, wherein a light transmittance of an aqueous solution of the composition containing black ginger extract to light having a wavelength of 660 nm is 95% or greater, where a total amount of the 6 polymethoxyflavones in the aqueous solution of the composition containing black ginger extract is adjusted to 0.05 mg/mL.

<3> A composition for oral administration including the composition containing black ginger extract according to <1> or <2>.

### INDUSTRIAL APPLICABILITY

Since the composition containing black ginger extract has the improved taste from the black ginger extract and the improved solubility to water and an acid solution, the composition containing black ginger extract can be widely used as cosmetic products, beverages and food, medical and pharmaceutical products, regardless of fields. Moreover, the composition containing black ginger extract is suitably used for the below-described composition for oral administration of the present invention.

Since the composition for oral administration of the present invention includes the composition containing black ginger extract, moreover, the composition for oral administration can be suitably used for nutritional enhancement, such as increasing energy, enhancing sexual performance, improving symptoms of stomach or bowel illness, and improving pain, or for anti-oxidation, anti-aging, anti-inflammatory, hair growth, anti-obesity, skin-brightening, etc.

1. A composition containing black ginger extract, comprising:
black ginger extract; and
cyclodextrin,
wherein the composition containing black ginger extract includes
6 polymethoxyflavones including 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, and 3,5,7,4′-tetramethoxyflavone,
when a total amount of the 6 polymethoxyflavones in the composition containing black ginger extract is determined as 100 parts by mass, an amount of 5-hydroxy-7-methoxyflavone in the composition containing black ginger extract is 5 parts by mass or less,
when absorbance of an aqueous solution of the composition containing black ginger extract, in which a total amount of the 6 polymethoxyflavones is adjusted to 0.006 mg/ML, is measured, the absorbance of the aqueous solution satisfies Formula (1) below:
P1/V1≥1.45 Formula(1)
where, in Formula (1), V1 is the minimum value of absorbance in a wavelength range of from 230 nm to 240 nm, and P1 is the maximum value of absorbance in a wavelength range of from 260 nm to 270 nm.

2. The composition containing black ginger extract according to claim 1, wherein a light transmittance of an aqueous solution of the composition containing black ginger extract to light having a wavelength of 660 nm is 95% or greater, where a total amount of the 6 polymethoxyflavones in the aqueous solution of the composition containing black ginger extract is adjusted to 0.05 mg/mL.

3. A composition for oral administration comprising the composition containing black ginger extract according to claim 1.
